(19) United States
(12) Patent Application Publication
(10) Pub. No.: US 2024/0424084 A1
(43) Pub. Date: Dec. 26, 2024

(54) UNDIRECTED MUTATED MRNA VACCINE

(71) Applicants: Dennis R. BURTON, La Jolla, CA (US); Douglas T. GJERDE, Saratoga, CA (US)

(72) Inventors: Dennis R. BURTON, La Jolla, CA (US); Douglas T. GJERDE, Saratoga, CA (US); David P. HORNBY, Widnes (GB)

(21) Appl. No.: 18/683,880

(22) PCT Filed: Aug. 13, 2022
(86) PCT No.: PCT/US2022/040272

§ 371 Date: Feb. 15, 2024

Related U.S. Application Data
(60) Provisional application No. 63/233,320, filed on Aug. 15, 2021.

(51) Int. Cl. A61K 39/215; A61K 39/00; A61K 39/02

(52) U.S. Cl. CPC A61K 39/215; A61K 39/0002; A61K 39/001102; A61K 39/02; A61K 2039/53; C12N 2770/20034

(57) ABSTRACT

We claim vaccines and a method of making vaccines targeted against diseases caused by viruses, including influenza virus and SARS CoV-2, against cancer, and diseases caused by bacteria, fungi, and other biomaterials/diseases that are combatted with an immune response. The mRNA vaccine is injected into the body whereupon the injected mRNA hijacks the translational machinery of the cells to produce an antigen such as a virus spike protein or surface protein (or part thereof) and stimulates an immune response. The mRNA in the vaccine is a mixture of mRNAs and where at least one or more of the RNAs are undirected mutant variants of the parent mRNA. The vaccine is a poly vaccine and provides protection against multiple variants. The vaccine may comprise mRNA species encoding several random undirected mutations directed against unknown variants.

### BACKGROUND OF THE INVENTION

Each year, a new flu shot is developed to try to combat the strains of the influenza virus expected to wreak havoc in the next season. Virus strains change year by year through spontaneous mutation. Natural levels of viral genome mutation are often associated with antigenic drift. These are small changes (or mutations) in the genes of influenza viruses that can lead to changes in the sequences of surface proteins of the virus that are the target for antibodies: viral mutations can have positive effects, deleterious effects, or no effect on the ability of the virus to survive and spread. 142 national influenza centers in 113 different countries collect data on the flu viruses known to be present that year in specific localities. Information is collected on the particular virus strains infecting these isolated populations and how those strains are spreading. This information is analyzed by epidemiologists to determine which strains of the virus are most likely to spread into the world population in the next flu season. Then a working vaccine against these strains is developed. Coronavirus may also mutate and develop antigenic drift although owing to differences in genome structure and regulation, the antigenic drift rates are correspondingly different. Cancer is the result of mutations that cause cells to proliferate and spread in a fashion avoiding normal homeostatic control. Cancer cells may also mutate as they divide in the body.

Vaccines work by exposing the body to molecules called antigens, which trigger an immune response. In traditional vaccines, antigens are presented on attenuated or killed virus or fragments of virus. Under normal circumstances, when the body senses these foreign antigens, it kick-starts specialized immune defenses, including the activation of immune cells known as lymphocytes, which deploy systems to eliminate invaders. Following elimination, the body stores a “memory” of that particular invader for years or even decades, guarding against future infections.

A commonly used method to produce influenza vaccine involves injecting live viruses into a fertilized chicken egg, then letting the egg incubate and become infected with the virus. Afterwards, the liquid inside the egg is removed and mixed with an embalming fluid called formalin. It renders the virus incapable of causing an infection but still capable of provoking an immune response that will protect vaccine recipients against future infections.

The development and manufacturing of mRNA for use as therapeutics and vaccines are now perceived as comparatively simple, scalable, and extremely rapid. With a compressed timeframe from development to clinic and towards approval, mRNA technology is attractive not only for response to outbreaks of infectious diseases and pandemics, but also for development of novel therapeutic approaches for addressing diseases with unmet needs. The transformational, early results for the commercial mRNA vaccines against SARS-CoV-2 have implications that go far beyond the current pandemic and bode well for similar approaches in the fight against other viral infectious diseases, cancer, heart disease and wide range of microbial infectious diseases. Vaccines of the invention apply to all of these areas.

In contrast to older vaccine technologies, mRNA vaccines protecting against viral infections are produced by manufacturing the virus RNA that codes for the protein spikes of the virus. mRNA is encapsulated in lipids and injected into the body. Lipids help deliver the mRNA into the body's cells and the mRNA is released into the cytoplasm. In the cell, the RNA hijacks the machinery of the cells to produce the virus protein spikes. The body's response to these proteins is the same as with earlier types of vaccine. An immune response is elicited so that future infections are repelled.

Every vaccine must be thoroughly tested and approved by the FDA before it is made available to the public. In many years, due to the time required by the process to make each new vaccine, the process results in a flu vaccine that does not fully protect against the flu. In some years, the vaccine does not protect at all against the actual strains of flu present in the general population. And there are fears that the current mRNA vaccines will not protect the population against new emerging mutated strains of COVID-19, or as they are called, variants of concern.

Broadly speaking, neutralization of a virus is achieved when something like 50% of the spike proteins are occupied by antibody. The antibody molecule (150 kD) is actually quite large compared to typical spike proteins and once an antibody is attached then entry tends to be inhibited. This is not only by blocking attachment or fusion but also by inducing disassembly of spikes.

There exists a need to improve vaccine design and production so that better protection is achieved, even against a new virus or new cancer cells introduced by antigenic drift and other natural mutations.

### DETAILED DESCRIPTION OF THE INVENTION

We claim vaccines and a method of making mRNA vaccines targeted against infection by viruses, including influenza virus and SARS CoV-2, against cancer, infection by bacteria, fungi, and other biomaterials/diseases that are combatted with an immune response. The mRNA formulated in the vaccine is a mixture of mRNAs and where at least one or more of the RNAs are undirected mutant variants of the parent mRNA. The parent mRNA is the starting material used for generating undirected mutations. The vaccine is a poly vaccine and provides protection against multiple variants. The vaccine may comprise mRNA species encoding several random undirected mutations directed against unknown variants. Vaccines of the invention may produce a poly efficacy; however, the undirected mutations in the vaccine of the invention are derived from a wild type or parent with known disease proteins and a known coding sequence.

Undirected mutated mRNA is any mRNA molecule that is not directed or coded to produce a protein antigen of a known disease variant. An undirected mutated mRNA is any mRNA molecule that is mutated from a known sequence coding for known protein antigens (also called wild-type antigens). Undirected mutated mRNA vaccines produce the corresponding mutated protein antigens to these known antigens.

Stated another way, in some embodiments of the invention, the vaccine of the invention contains the mRNA sequence of the infectious virus to which the vaccine is directed. However, the vaccine also contains and comprises mutated forms of the wild type mRNA sequence (including ancestral mRNA). For the purpose of this invention wild type sequences include sequence variants that were known variants, subsequent to the original nucleic acid sequence that are known to cause disease. Undirected mutations can be made from these wild type or parent sequences. In this way, a mixture of closely related mRNA molecules is introduced into the body. The vaccine mRNA is typically injected and enters the cells, whereupon the protein of the virus is produced and an immune response to the protein is generated. However, the vaccine also contains a mixture of variants of the mRNA containing one or more mutations. Thus, the immune response generated is not for a protein produced by a single type of mRNA, but the immune response is for many different, but closely related protein variants. In some embodiments of the invention this includes one or more mRNAs coding for a known variant or known variants.

The introduction of mutations into genetic material (which may be DNA or RNA) encoding an open reading frame, such as a viral spike protein, can be achieved in several ways. Site directed or rational mutagenesis, is the process in which mutations are introduced in a sequence-specific manner to specific locations in the genetic material (often a gene or part thereof incorporated into a plasmid or viral vector). Such mutations may be single or multiple nucleotide changes, usually made at strategic locations throughout the nucleotide sequence encoding the open reading frame. These changes can be introduced for example, by incorporation of mismatched oligonucleotides in conjunction with a suitable DNA polymerase, or simply by substituting segments or the complete nucleotide sequence, encoding any gene of interest, with a chemically synthesized double-stranded sequence. In the case of mRNA vaccines, amino acid coding substitutions are made in this way and individually prepared mRNA sequences are obtained following in vitro transcription from the mutant template. This is the current method used to develop new mRNA vaccines.

In some embodiments of the invention, vaccines are produced from a mixture of mutated mRNA molecules. Undirected mutagenesis is defined as the introduction of changes to include base transitions, transversions, deletions and insertions. The changes may be introduced chemically, enzymatically, or biologically. In some embodiments of the invention mutations into a specific nucleotide sequence are made without prior consideration of the structural or functional consequences of the impact of such changes. In some embodiments of the invention, mutations are made into a specific nucleotide domain that may be known to be structural, such as a spike protein or a portion of a spike protein, but without prior knowledge of the structural or functional consequences of the impact of such changes. The density of the undirected mutations is not necessarily uniform across the undirected mutated mRNA. The density of mutations may be controlled to ensure coding for a variety of protein structures.

Undirected mutations include any mutation that is not directed to a specific, known or discovered variant of the primary developed vaccine. In some embodiments of the invention, the mutations are directed to include conserved regions of the sequence. In some embodiments of the invention, the mutations are directed to included un-conserved regions of the sequence.

In an example of an embodiment of the invention, undirected mutagenesis generated an mRNA vaccine containing between 1 and 10, 1 and 100 or 1 and 1000 point mutations. In some embodiments of the invention the undirected mutated mRNA contains at least 1, at least 50, at least 100 or at least 1000 undirected mutations. These three mixtures are formulated into one or more vaccines. When a mixture of such variant mRNAs is introduced into the body, the vaccine will produce a range of proteins to which antibody protection is established. In certain embodiments, the introduction of mutations may be restricted to specific segments (or domains) of the sequence encoding the spike protein: this is referred to as site-specific or region-specific undirected mutagenesis. This is defined as undirected mutagenesis applied to sub-regions of a given gene sequence. It can be achieved in several ways including undirected mutagenesis of a restriction fragment encoding a subsection of a given gene, or by substituting segments of coding sequence previously exposed to undirected mutagenesis.

The number of variants in the mRNA population so produced from amplification of the complete coding sequence may range from 1-100 or 100-1000 or 1000-1000000. The region of sequence mutated may also be further defined. For example, the sequence encoding the receptor binding domain in a virus or cell may be amplified by the above error-prone methods, thereby limiting variants in the vaccine accordingly. In one example, this can be achieved by selective error-prone amplification of a double stranded DNA fragment encoding the receptor binding domain segment, followed by substitution of this segment into a suitable plasmid encoding the remaining (flanking) sequences of the spike protein mRNA. This will generate a library of receptor binding domain (RBD) targeted variants, providing a template for mRNA transcription. This could also be achieved by chemical synthesis of a library of oligonucleotide duplex variants.

The protection afforded by this form of vaccine, is produced by an immune response to not only one protein but several, or many different, but related proteins. In this way, the viral mutations are anticipated by the immune system, and protection is achieved before circulating (atmospheric) virus mutates into different strains.

In another example of an embodiment of the invention, the cDNA encoding the viral spike protein may be subjected to undirected mutagenesis by amplification with a DNA polymerase under error-prone conditions, and the variant population combined to act as a template for in vitro transcription by a recombinant RNA Polymerase, such as the bacteriophage T7 RNA Polymerase. The final vaccine preparation will contain multiple copies of variant mRNAs. It is also possible to introduce undirected mutations directly into the mRNA sequences through the replacement of the high-fidelity RNA polymerases conventionally used to synthesize mRNA in vitro, with error-prone variants of the polymerase. In this way the preparation of variant mRNAs for vaccine production may be simplified.

In another approach, the gene encoding the complete ORF of the SARS-CoV-2 receptor binding domain (RBD), appended with a T7 RNA polymerase promoter and ribosome binding site, or alternatively, the nucleotide sequence specifying just the coding sequence is amplified using an error-prone DNA polymerase. The variant library of sequences is used to feed an in vitro transcription reaction. The “library” containing up to thousands of versions of the encoded mRNAs would contain different mutations and so encode receptors with slightly altered amino acid sequences, which in turn may encode proteins possessing the same, similar or different properties than the original mRNA sequence such as, for example, a template encoding the Wuhan variant spike protein or known variants.

It might be argued that immunization with a library of antigens will only generate immune responses to a small fraction of the antigens and therefore will not be useful in producing effective responses to newly emerging variants. Immunodominance is a well-known phenomenon in immunology whereby responses against different antigens and epitopes are very unequal. Another problem in immunology is that once responses have been made to a given antigen then it can be difficult to raise antibodies to closely related antigens. This phenomenon is known as original antigenic sin or imprinting. It is believed to arise because the variant antigens stimulate memory responses to the original antigen rather than de novo responses to the new variant antigen. However, random mutations, especially mutations that are directed to specific domains will eventually produce significant protein structural changes, just as mutations in the wild may change protein structure sufficiently to require a new version of the vaccine. Certain mutations causing significant protein structural changes are much less likely to be affected by immunodominance or imprinting.

Finally, the amount of antibody that can be secreted into plasma to fight viruses is limited by the population of antibody-secreting cells that can be accommodated in the bone marrow niche. Thus, the number of antibody specificities that can be generated is not limitless. However, we expect that the number of variants to be deployed at any time will not exceed the capacity of the bone marrow to generate appropriate antibodies.

Many possible antibodies can be raised against any viral antigen introduced to the body. The result can be thousands of different antibodies that bind to different proteins or different parts of the same protein of a virus, bacterial cell, fungal cell, cancer cell or spore or other surface protein of a diseased cell or infection-causing agent.

This strategy is effectively like immunizing individuals with a poly-clonal antigen. In the example where the entire coding sequence is subject to mutagenesis, no assumptions are made about the primary structure determinants likely to elicit an immune response. Even in the example where mutations are directed to known regions, the immune response remains unpredictable. But so too is the progression of a disease, driven by new variants including new variants of cancer cells, etc. Nevertheless, undirected mutated vaccines can offer protection to new disease variations that may be presented.

Making the mRNA

The mRNAs can be synthesized chemically, but they can also be synthesized enzymatically. In some embodiments, the mRNAs are purified at scale using a form of ion pair reverse phase chromatography, eluted in acetonitrile and cleaned up for injection.

Production of mRNA-based therapeutics and vaccines typically or commonly begins with a pDNA template that contains a DNA-dependent RNA polymerase promoter and the corresponding sequence for the mRNA construct. Given the central role of the pDNA construct, its design and purity are important factors for optimizing the mRNA product. pDNA production and purification present several challenges due to the large size of the nucleic acid and its high viscosity, shear sensitivity and the similarities between the pDNA and impurities.

The mRNA construct is designed to ensure efficient expression of the gene of interest. Stability, gene expression and efficient protein translation depend upon several structural elements The mRNA structure:
The cap region at the 5′ end of the sequence is essential for mRNA maturation and allows the ribosome to recognize the mRNA for the efficient protein translation. The cap also stabilizes mRNA by protecting it from nuclease digestion.
The untranslated regions (UTRs) located at the upstream and downstream domains of the mRNA coding region are affecting translation efficiency, localization and stability and can be utilized for efficient protein expression.
The open reading frame or coding sequence regions contains the gene of interest (GOI).
The poly-(A) tail is crucial for protein translation and mRNA stability by preventing digestion by 3′ exonuclease.

The required pDNA is amplified within bacterial cells, such as E. coli, and subsequent purification steps yields a pure, concentrated, circular pDNA. The pDNA is then linearized to serve as a template for the RNA polymerase to transcribe the desired mRNA.

Linearization is required to avoid transcriptional read through events that may generate undesired forms of mRNAs leading to additional impurities that would need to be removed. Linearization is achieved by mixing the plasmid DNA with a restriction enzyme in a reaction buffer and subsequent incubation at 37° C. for 4 hours. Optionally, the reaction is stopped by the addition of EDTA or heat inactivation at 65° C.

Impurities such as the restriction enzyme, BSA, DNA fragments, endotoxins and others can then be removed. Most of the lab scale processes use a solvent extraction technique and this not applicable for GMP production environments.

As an alternative, tangential flow filtration (TFF) and chromatography are efficient impurity removal techniques for this purification step.

The next step is in vitro transcription during which the linearized pDNA, serving as the DNA template, is transcribed into mRNA. This enzymatic reaction uses elements of the natural transcription process, including RNA polymerase and nucleotide triphosphates. Following transcription, the final mRNA structure requires a 5′ cap structure for stability and efficient transduction in the cell.

The cap can be added co-transcriptionally or enzymatically. Co-transcriptional capping is usually accomplished by adding cap analogs and guanosine triphosphate (GTP) in the transcription mix at a ratio of four cap analogs for one GTP. Following an incubation step at 37° C., the DNA template is typically degraded by the addition of DNases; the resulting small DNA fragments can then be easily separated from larger mRNA molecules by tangential flow filtration (TFF). Another option to remove the DNA template includes the utilization of a chrome step (e.g., Poly (dT) capture). In the latter case the DNA template does not need to be digested, which avoids the risk of small DNA fragments hybridizing to the mRNA.⁴

Co-transcriptional capping is less expensive and faster than enzymatic capping as it is performed during the transcription step, in the same reactor mix. However, efficiency and yield are lower, and it can generate non-capped impurities as GTP can bind to the mRNA sequence instead of the cap analogs. In addition, the cap analogs can be incorporated in the reverse orientation. To overcome this, some anti reverse cap analogs (ARCA) have been developed to prevent this reverse incorporation of a 5′ cap, leading to higher translation efficiency.

Enzymatic capping is performed after mRNA purification from the in vitro transcription mixture. This reaction usually uses a vaccinia virus-capping enzyme to add the capping structure to the mRNA structure. While enzymatic capping has a very high capping efficiency, it is more expensive and requires an extra unit operation.

### Methods of Mutating Nucleic Acid to Produce Undirected Mutations:

In some embodiments of the invention, undirected mutations are introduced in the pDNA template used to produce the mRNA.

Error-prone PCR uses a low-fidelity or “sloppy” version of PCR, in which the DNA polymerase has a significantly higher error rate that yields mutant amplicons exhibiting mutation frequencies of between 1-20 substitutions per 1000 bp. The PCR can be made error-prone in various ways including increasing the MgCl₂ in the reaction, adding MnCl₂ or using unequal concentrations of each nucleotide. Other strategies and compounds can also be used decrease fidelity.

After amplification, the library of mutant coding sequences can be used directly as a template for mRNA production, or ligated into a suitable plasmid vector, which is then amplified in vivo and purified in advance of an in vitro transcription reaction. Although point mutations are the most common types of mutation in error-prone PCR, insertions, deletions and frameshift mutations are also possible.

Rolling circle error-prone PCR is a variant of error-prone PCR in which a wild-type nucleotide sequence is first incorporated into a plasmid vector, then, as above, the whole plasmid library is amplified under error-prone conditions. This eliminates the ligation step that limits library size in conventional error-prone PCR, but the amplification of the whole plasmid is less efficient than amplifying the coding sequence alone.

Chemical mutagens such as ethyl methane sulfonate (EMS) can be used. EMS alkylates guanidine residues, causing them to be incorrectly copied during DNA replication. Since EMS directly chemically modifies DNA, EMS mutagenesis can be carried out either in vivo (i.e., whole-cell mutagenesis) or in vitro. An example of in vitro mutagenesis with EMS in which a PCR-amplified gene was subjected to reaction with EMS before being ligated into a plasmid and transformed.

Nitrous acid is another chemical mutagen. It acts by de-aminating adenine and cytosine residues (although other mechanisms are possible) causing transversion point mutations (A/T to G/C and vice versa).

DNA Shuffling is a very powerful method in which members of a gene library (i.e., copies of the same gene each with different types of mutation) are randomly shuffled. This is done by randomly digesting the library with DNase then randomly re-joining the fragments using self-priming PCR.

High density undirected mutations can be introduced for a short sequence and lower density mutations can be introduced for other parts of the sequence; we refer to this as the distribution of mutations. In some embodiments, the distribution of mutations can be more or less uniform along the sequence. Mutations may be introduced for every 2-10 bases, 5-10 bases, 5-20 bases or at a lower rate, for example every 20-50 bases or 50-100 bases. Sequences with different rates of mutation may be combined systematically or in an undirected way, to produce the code for the mRNA to be produced and subsequently translated in vivo. Specific domains or nucleic acid regions may be known to be critical to disease. These include nucleic acids that code for spike proteins or other types of surface proteins for different diseases including those caused by cancer, bacteria, fungus, etc. Undirected mutations may be performed or focused to a specific domain or nucleic acid sequence region.

Mutations may be synthesized to contain different nucleotides, insertions and/or deletions and then linked together with a known sequence. A sequence generated from these synthesized mutations is still considered an undirected mutated sequence because the effectiveness of a vaccine made from these sequences is unknown. Even though the exact mutation sequence may be synthesized and known, the mutation is defined to be an undirected mutation provided it is not a sequence of a known disease in the public.

Different mutated domains may be linked together or placed into the final sequence, each having different mutation densities. Or the entire sequence can be mutated to different mutation densities.

Sequences encoding mutations that are known to produce virulent variants may be introduced to increase the effectiveness of a vaccine to some future threat. However, the undirected nature of the vaccine is what makes it powerful to a future threat. The virus may randomly mutate to a virulent and dangerous form. But it is unknown what mutations actually produce a dangerous variant. The mutations are not directed in nature and the chances of a mutation being dangerous are low. However, there is a chance that a particular mutation can be more dangerous or may be able to evade the protection offered by a classical mRNA vaccine. The vaccine i.e., the RNA can be randomly mutated to meet that threat. In fact, random mutations may be particularly effective because it resembles evolution that is dramatically speeded up to meet the threat before the naturally mutating virus can produce the dangerous variant.

mRNA production for the use in vaccines, can be performed in a one-step enzymatic reaction, where a capping analog is used, or in a two-step reaction, where the capping is performed using a vaccinia capping enzyme.

Functional synthetic mRNA may be obtained by in vitro transcription of a cDNA template, typically plasmid DNA (pDNA), using a bacteriophage RNA polymerase. Hence, the preparation of pDNA is the first step in the production of mRNA.

Error-prone polymerases may be used to generate the random mutations. In normal PCR, the polymerase and conditions are selective to create a high fidelity copy i.e., exact copies of the original template. Error-prone polymerase is a polymerase that has been engineered to purposely produce low fidelity copies of the nucleic. The resulting mixture of products contain random errors or mutations in reproduction. The number of errors per number of bases increases with PCR cycle number. Thus, the number of mutations within a target sequence can be controlled.

mRNA is produced by in vitro synthesis through an enzymatic process; this contrasts with classical in vivo protein expression where time-consuming cloning and amplification steps are needed. Because an in vitro synthesis process is used, there is no need to remove cells or host cell proteins. This simplified manufacturing process, which uses the same reaction materials and vessels for any target, allows GMP facilities to switch to a new protein target within a very short period of time, with minimal adaptation to process and formulation.

Methods for the Production of Populations of Undirected Mutant mRNA Sequences Encoding Viral Spike Proteins, for Vaccination:

Any low fidelity DNA or RNA polymerase can be used with the methods of the invention. In one example, pDNA encoding part, or all of the viral spike protein, incorporating a bacteriophage T7 promoter sequence at the 5′ end, is used as a template for error-prone PCR. In one approach, the method described by Biles and Connolly may be implemented to introduce undirected, point mutations into the PCR reaction products (Benjamin D. Biles, Bernard A. Connolly, Low-fidelity Pyrococcus furiosus DNA polymerase mutants useful in error-prone PCR, Nucleic Acids Research, Volume 32, Issue 22, 15 Nov. 2004, Page e176). Following, amplification, the enzymatically synthesized DNA variant sequences are recovered from the PCR reaction mixture and purified by standard methods (see Sambrook, J., Fritsch, E. R., & Maniatis, T. (1989). Molecular Cloning: A Laboratory Manual (2nd ed.). Cold Spring Harbor, NY: Cold Spring Harbor Laboratory Press.)

The library of variant DNA sequences, so produced, is then added to a commercial mRNA synthesis reaction, such as New England Biolabs' HiScribe™ T7 ARCA mRNA Kit (with tailing), following the manufacturer's instructions (https://international.neb.com/products/e2060-hiscribe-t7-area-mrna-kit-with-tailing#Product %20Information.)

The population of mRNA transcripts synthesized in this way, encode a range of undirected variants, reflecting those mutations introduced in the error-prone PCR step. The mRNAs are also, suitably modified for subsequent in vivo expression in the host, following administration of the vaccine.

The generation an mRNA library of variants may also be achieved using a combination of chemical and enzymatic DNA and RNA synthesis. In addition, the direct synthesis of variant mRNAs can be achieved using a low-fidelity RNA polymerase, thereby obviating the need for the error-prone PCR step.

The population of mRNA variants may then be purified, for example, by preparative, ion pair reverse phase chromatography. The population of solvent-free mRNAs is now ready to be formulated into the final vaccine.

Purifying mRNA

Following the in vitro transcription step, mRNA is purified from the impurities and materials used in the previous steps including endotoxins, immunogenic double stranded RNA (dsRNA), residual DNA template, RNA polymerase and elemental impurities.

mRNA purification can be accomplished using a variety of methods. For example, tangential flow filtration (TFF) allows efficient separation of mRNA from smaller impurities that are not retained by the membrane; molecular weight cut-offs ranging from 30 to 300 kDa can be used based on the size of the mRNA.

A number of chromatography techniques can be used as an alternative to TFF and include reverse-phase ion pair, anion exchange and affinity chromatography using poly(dT) capture.

Chromatography provides an efficient means for DNA template removal and eliminates the risk of hybridization that can occur during Ultra-/Diafiltration step. It is, however, more expensive and a TFF step would still be required for media exchange and preparation for the subsequent step.

Chromatography can also be also used following the enzymatic capping step to remove unwanted products and oligonucleotide impurities coming from the previous enzymatic reaction steps.

Reversed-phase ion pairing is commonly used at small scales and allows a very efficient and rapid RNA purification and good separation of single stranded RNA (ssRNA) from DNA, double stranded RNA (dsRNA), and short transcripts. The technique requires ion-pair reagents and resulting formation of complexes with the mRNA may require extensive diafiltration steps for removal.

Anion exchange has a high dynamic binding capacity and is very efficient for removing immunogenic impurities such as dsRNA, uncapped RNA, RNA-DNA hybrids and other RNA structures. While this allows the use of aqueous solutions, it might require the addition of chaotropic agents that can be toxic and operation at temperatures of up to 85° C. to desorb large mRNA molecules bound to the resin. Ambient temperature operations typically elute mRNA species smaller than 500 bases.

Affinity chromatography poly(dT) capture uses a resin to specifically capture the poly(A) tail of full-length mRNA transcripts. This process efficiently removes DNA, nucleotides, enzymes, buffer components and any other impurities not having a poly(A) tail.

The initial chromatography step can be affinity chromatography typically followed by a second chromatography step using anion exchange for polishing purposes.

Following the chromatography step(s), a final concentration and diafiltration is performed to maximize product purity and transfer the mRNA into the appropriate buffer for formulation or storage. At this stage, mRNA can be further purified, and concentrated. A sterile filtration step can be performed following this TFF step.

Formulating the mRNA

After generating a population of undirected mutated mRNA sequences, they can be formulated into a vaccine. For example, mutagenesis can be performed using a viral spike protein as a template or parent sequence. In some embodiments, the vaccine is comprised of a mixture of different undirected mutated mRNA sequences. The mixture can include different sequences generated from a single parent sequence or the mixture can be made up of sequences generated from more than one parent.

mRNA vaccines may be delivered via lipid nanoparticles Some lipid (fat) molecules have an interesting property: one end of each molecule is attracted to water molecules (hydrophilic), and the other end is repelled by them (hydrophobic). Pfizer BioNTech and Moderna and many other companies and researchers have published on mRNA formulations to shield and allow mRNA to enter the cell.

mRNA vaccine technology may incorporate, non-viral delivery systems and offer considerable versatility, obviating the requirement for the lengthy preparation of biochemically purified, protein antigens. Delivery of an mRNA into the cytosol of a cell can induce the production of a target protein which in turn can function as a therapeutic or prophylactic, act as an antigen to trigger an immune response for vaccination purposes, replace a defective protein or activate an anti-tumor response.

Delivery tools are equally important in the effectiveness of mRNA vaccines and therapeutics. After the final mRNA purification step, the next consideration is the delivery mechanism. One of the most advanced classes of delivery systems are combinations of lipids and polymers. These include complexes of oligonucleotides bound to lipids forming a lipoplex or positively charged polymers such as polyethyleneimine (PEI) forming polyplexes.

Lipid nanoparticles (LNP) are most commonly used for mRNA delivery; each lipid nanoparticle consists of four different lipids allowing the mRNA to be carried in it and protected from degradation. These lipids are also responsible for efficient release of the RNA into the cytoplasm. The structure of cationic lipids has a major impact on the activity of the LNP, its toxicity and effectiveness.

Lipids should be chosen based on the delivery route in mind to achieve maximum efficacy and optimal biodistribution. In addition to the choice of lipids, the ratio between the individual lipids it is an important component to finetune, as it has a direct impact on the bilayer fluidity and the effectiveness of the LNP.

Several critical aspects must be considered when selecting the lipid. Lipid type, source and quality have a direct impact on the impurity profile and properties such as the particle characteristics, stability and release profile are the final formulation. To achieve reproducible results with the final formulation, consistent quality of lipids is required, which is dependent on the quality of the raw materials used to synthesize the lipids and appropriate material characteristics of the lipid itself.

The purified mRNA can be formulated into the delivery particle via different techniques. In the commonly used solvent injection technique, lipids are dissolved in a solvent such as ethanol and rapidly mixed in an aqueous, low pH buffer containing the mRNA using a crossflow mixing or microfluidic mixing is to create the LNPs. The low pH buffer is then filtered into a neutral buffer and ultrafiltration is used to concentrate the particles.

When mixed with water and other molecules, such as RNA, the hydrophobic regions will coalesce around the RNA forming nano-scale spheroids with the hydrophilic ends on the outside. These nanoparticles can penetrate biological cell membranes, and thereby deliver the encapsulated mRNA, which in turn is made available for ribosomal protein synthesis.

Tangential flow filtration purification after formulation may be used but must be rapid as lipids can be hydrolyzed at low pH, leading to formation of impurities such as hydrolipids that can affect the lipid bilayer structure, stability of the formulation and drug release characteristics. Degradation of the lipids can also increase the size of the particle, resulting in aggregation.

LNPs have a very good stability, structural plasticity and enhanced gene delivery compared to other delivery systems. They increase the transfection rate compared to naked mRNA, allow for intravenous injection without the risk of being degraded by RNases present in the bloodstream and enable active targeting if specific ligands are incorporated.

Disadvantages of LNPs include the fact that they may require cold chain logistics. In addition, sterile filtration is not always possible with LNPs and in such cases alternatives, such as gamma irradiation, heat sterilization, high-pressure sterilization or closed processing must be considered.

### Possible Domains for Undirected Mutation:

In some embodiments of this invention, the entire or substantial sequences of mRNA or DNA may undergo undirected mutagenesis. In some embodiments of this invention, specific domains or sequences may be targeted for undirected mutagenesis. In some embodiments, varying amounts of undirected mutagenesis may be administered for different sequence segments. In some embodiments of the invention, RBD region(s) or sites in the RBD region(s) are preserved or conserved and other regions of the sequence undergo undirected mutation. In other embodiments, the receptor binding domain can undergo undirected mutagenesis while the rest of the viral genome is conserved.

Viral proteins tend to be different sizes and shapes. The overall size of a receptor binding protein might be for example 100 kD but the actual crucial residue binding footprint will be much smaller. Critical binding sequences are distributed quite widely in the linear sequence. In some embodiments of the invention, particular regions of interest may be targeted for undirected mutation. The sequences may be several short regions, maybe 10 amino acids or so) and mutation may be confined to those regions. Mapping of proteins has been performed (see e.g., https://www.nature.com/articles/s41586-022-04464-z.)

The target for vaccines in the case of an enveloped virus is often a single protein (which can be a spike or envelope protein) that attaches to a cell-surface protein/receptor on the surface of a particular host cell. For some viruses, attachment and fusion of the virus with the host cell membrane to permit entry of viral nucleic acid to the host cell is mediated by a separate fusion protein. This could include the complete sequence of the spike or fusion protein surface exposed sequences of the spike or fusion protein, all sequences specifically involved in receptor binding or fusion, strategically identified sections of the spike or fusion protein that are critical for receptor binding. The length of the polypeptide sequence reduces as you home in on the receptor binding region or the fusion region.

Flu and HIV and SARS2 use single entry proteins but other viruses can use a fusion protein e.g., RSV, respiratory syncytial virus. Still other viruses are more complex and use multiple proteins to gain entry e.g., herpes viruses and then most complex, pox viruses. RSV vaccine is based on targeting the fusion (F) protein rather than the attachment (G) protein.

Our goal in this invention is to generate a selection of mRNA immunogens that will, when administered together, induce an array of antibodies that will anticipate mutations in the virus and so prevent the emergence of escape variants or variants of concern. The selection of mRNAs will be generated by random mutagenesis to targeted regions of the viral surface proteins (including spike proteins) that are recognized by neutralizing antibodies (nAbs). These regions can be identified from knowledge of the structures of nAb-spikes that are determined by X-ray crystallography, cryo-electron microscopy (cryoEM) or newly developed techniques of artificial intelligence (AI), such as Alpha Fold. The regions to be identified will include contact regions with nAbs but also regions that can be inferred to allow escape from nAbs. Methods to identify regions that contact proteins and might perturb protein-protein interactions have been developed e.g., https://www.nature.com/articles/s41586-022-04464-z.

Once critical regions have been established, mRNA of the spike protein of the example will be randomized in the key nAb footprint and sensitive areas. Mutations that are deleterious to protein generation and therefore unlikely to be incorporated into virions will not be expressed and therefore will not interfere with immunization. Hotspots of Ab recognition typically only include 5-10 amino acid residues that will be the first focus of sequence randomization. As understanding of nAb recognition develops, randomization could be extended to further residues.

An added advantage of this approach is that once strong nAb responses to a particular set of residues in a given site have developed then escape will tend to occur by mutation in some of those residues. On vaccination with the mRNA cocktail, the pre-existing response to the set of residues will clear the corresponding immunogen from the cocktail and immunogen expressing potential escape mutations will tend to predominate. This will allow productive vaccination of individuals who have already been vaccinated with a single mRNA or had a relevant infection e.g., vaccinated with SARS-CoV-2 mRNA vaccine of the ancestral Wuhan strain or infected with SARS-CoV-2. In the case of COVID-19, as most people have now been infected or vaccinated, this could be an important consideration.

### A Type of Undirected Mutated SARS-CoV-2 Vaccine:

The sequence encoding the SARS-CoV-2 spike glycoprotein was chosen by both Moderna and Pfizer-BioNTech to generate their vaccines in the form of mRNA. There are chemical differences between the two mRNA species (Pfizer incorporated pseudo-uracil to enhance translational efficiency, while Moderna used standard nucleotides. However, there are codon optimization differences, these are mainly aimed at optimizing translational efficiency. Specific amino acid substitutions were also incorporated to reflect conformational differences that were anticipated to influence the nature of the immune response.

The full length, SARS-CoV-2 spike glycoprotein (the major envelope protein of the virus), in conjunction with proprietary lipid nanoparticle preparations form the basis of the vaccine.

In one embodiment of the invention, undirected mutation of the mRNA for incorporation into vaccines is based on the complete coding sequence for the viral spike protein. The sequences are optimized for translational initiation, elongation and termination, in order to ensure that when the mRNAs reach the ribosome, they are expressed with high efficiency. Sequences may also be altered by incorporating any alternative nucleotide chemistries e.g., pseudo uracil substitution of U.

The mRNA is formulated as per each manufacturer, but typically this includes some form of lipid nano-particle encapsulation.

Following vaccination with the single mRNA, an immune response is elicited (in its most general terms) and the patient is protected to a level determined by post immunization evaluation. This has proved highly successful.

This proposal is novel, because the strategy described is distinct from those used in the current development of mRNA vaccines, since while the mRNA encoding the spike protein, or parts thereof, forms the basis of the starting point for the vaccine, the immunizing agent comprises a serendipitous set of mRNA variants. These variants are derived from transcription of the initial mRNA with an error-prone nucleic acid polymerase. In one example, which we refer to as indirect variant generation, error-prone DNA polymerase amplification feeds a standard RNA polymerase, such as T7 RNA polymerase. Direct variant generation is possible by using any intrinsically error-prone RNA polymerase, mimicking the situation found in many RNA viruses.

1-15. (canceled)

16. A method of making an mRNA vaccine comprising:
(a) providing a parent nucleic acid sequence, wherein the parent nucleic acid sequence encodes a surface protein of an infection-causing agent or a cancer cell;
(b) performing undirected mutagenesis on the parent nucleic acid sequence to produce a variant population of undirected mutated sequences;
(c) transcribing the variant population of undirected mutated sequences to produce a variant population of undirected mutated mRNA sequences, wherein the variant population of undirected mutated mRNA sequences comprises at least one sequence that is not a known disease sequence; and
(d) formulating the variant population of undirected mutated mRNA sequences into a vaccine active against an unknown disease variant.

17. The method of claim 16, wherein the parent nucleic acid sequence encodes a viral spike protein.

18. The method of claim 16, wherein the mRNA vaccine is designed to induce an immune response to a cancer cell, and wherein at least one mutation in the variant population of undirected mutations is present in a sequence coding for a surface protein of the cancer cell.

19. The method of claim 16, wherein the mRNA vaccine is designed to induce an immune response to a bacterium or a fungus, and wherein at least one mutation in the variant population of undirected mutations is present in a sequence coding for a surface protein of the bacterium or the fungus.

20. The method of claim 16, wherein the mRNA vaccine is designed to induce an immune response to a virus, and wherein none of the mutations in the variant population of undirected mutations is present in a sequence encoding a receptor binding domain of the virus.

21. The method of claim 16, wherein the mRNA vaccine is designed to induce an immune response to a virus, and wherein at least one mutation in the variant population of undirected mutations is present in a sequence encoding a surface protein of the virus.

22. The method of claim 16, wherein the mRNA vaccine is designed to induce an immune response to a virus, and wherein at least one mutation in the variant population of undirected mutations is present in a sequence encoding a receptor binding domain of the virus.

23. The method of claim 16, wherein the parent nucleic acid sequence comprises a viral spike protein coding sequence incorporated in a plasmid.

24. The method of claim 16, wherein step (b) is performed with a low fidelity DNA polymerase or an error-prone RNA polymerase.

25. The method of claim 16, wherein the variant population of undirected mutated mRNA sequences is formulated into a vaccine using lipid nanoparticles.

26. The method of claim 16, wherein the variant population of undirected mutated mRNA sequences contains between 1 and 10 undirected mutations.

27. The method of claim 16, wherein the variant population of undirected mutated mRNA sequences contains at least 50 undirected mutations, at least 100 undirected mutations, or at least 1000 undirected mutations.

28. The method of claim 16, wherein the variant population of undirected mutated mRNA sequences has a density of mutations that is not uniform across the variant population of undirected mutated mRNA sequences.

29. The method of claim 16, wherein the vaccine comprises a mixture of variant populations of undirected mutated mRNA sequences.

30. An mRNA vaccine prepared by the method of claim 16.

31. The mRNA vaccine of claim 30, wherein the parent nucleic acid sequence encodes a viral spike protein.

32. The mRNA vaccine of claim 30, wherein the mRNA vaccine is designed to induce an immune response to a cancer cell, and wherein at least one mutation in the variant population of undirected mutations is present in a sequence coding for a surface protein of the cancer cell.

33. The mRNA vaccine of claim 30, wherein the mRNA vaccine is designed to induce an immune response to a bacterium or a fungus, and wherein at least one mutation in the variant population of undirected mutations is present in a sequence coding for a surface protein of the bacterium or the fungus.

34. The mRNA vaccine of claim 30, wherein the mRNA vaccine is designed to induce an immune response to a virus, and wherein none of the mutations in the variant population of undirected mutations is present in a sequence encoding a receptor binding domain of the virus.

35. The mRNA vaccine of claim 30, wherein the mRNA vaccine is designed to induce an immune response to a virus, and wherein at least one mutation in the variant population of undirected mutations is present in a sequence encoding a surface protein of the virus.
